(19) United States
(12) Patent Application Publication
(10) Pub. No.: US 2026/0335609 A1
(43) Pub. Date: Oct. 29, 2026

(54) GUIDEWIRE WITH AN ILLUMINATED CHANNEL

(71) Applicant: Boston Scientific Scimed, Inc., Maple Grove, MN (US)

(72) Inventors: Timothy Lawrence Rubesch, Blaine, MN (US); Michael Francis McGovern, San Diego, CA (US); Brice Lee Shireman, Maple Grove, MN (US)

(21) Appl. No.: 19/659,264

(22) Filed: Apr. 27, 2026

Related U.S. Application Data
(60) Provisional application No. 63/795,838, filed on Apr. 28, 2025.

(51) Int. Cl. A61M 25/09

(52) U.S. Cl. CPC A61M 25/09; A61M 2025/09008; A61M 2025/09066; A61M 2025/09133; A61M 2025/09166; A61M 2205/0222; A61M 2205/0238; A61M 2205/0266; A61M 2205/583

(57) ABSTRACT

In some aspects, the techniques described herein relate to a medical guidewire that includes: a coiled wire first tubular member having a plurality of coils and openings between adjacent coils of the plurality of coils and extending along a length of the medical guidewire; a braided wire second tubular member including a plurality of constituent wires braided together and openings between the constituent wires that are braided together, the braided wire second tubular member extending along the length of the medical guidewire; and a side-emitting optical fiber extending along the length of the medical guidewire.

### CROSS-REFERENCE TO RELATED APPLICATION

This application claims priority to U.S. Provisional Patent Application No. 63/795,838, filed on Apr. 28, 2025, entitled “GUIDEWIRE WITH AN ILLUMINATED CHANNEL”, the disclosure of which is incorporated by reference herein in its entirety.

### TECHNICAL FIELD

This disclosure relates generally to implantable medical devices and, in particular, to guidewires with an illuminated channel.

### BACKGROUND

A wide variety of intracorporeal medical devices have been developed for medical use, for example, intravascular use. Some of these devices include guidewires, catheters, and the like. Guidewires are currently being used in medical procedures to guide catheters, sheaths or other devices from a remote site to a surgical site. From a remote part of the body, a guidewire can be introduced into an artery or vein. The guidewire is then advanced through the vascular system to the target site where one or more other devices can be positioned to perform a medical procedure. The guidewire then functions as a rail for advancement of these devices.

### SUMMARY

In some aspects, the techniques described herein relate to a medical guidewire that includes: a coiled wire first tubular member having a plurality of coils and openings between adjacent coils of the plurality of coils and extending along a length of the medical guidewire; a braided wire second tubular member including a plurality of constituent wires braided together and openings between the constituent wires that are braided together, the braided wire second tubular member extending along the length of the medical guidewire; and a side-emitting optical fiber extending along the length of the medical guidewire.

Implementations can include one or more of the following features, alone, or in any combination with each other.

For example, the coiled wire first tubular member can be disposed within the braided wire second tubular member.

In another example, the side-emitting optical fiber can be disposed within the coiled wire first tubular member.

In another example, the constituent wires of the braided wire second tubular member can include the side-emitting optical fiber.

In another example, the side-emitting optical fiber can include a core and a cladding surrounding the core, wherein an interface between the core and the cladding includes deviations from smoothness that are configured to scatter light out of the optical fiber in a radial direction transverse to a longitudinal axis of the side-emitting optical fiber.

In another example, the coiled wire first tubular member can have a pitch that varies along the length of the medical guidewire.

In another example, the coiled wire first tubular member can have a thickness that varies along the length of the medical guidewire.

In another example, the braided wire second tubular member can have a picks per inch (PPI) that varies along the length of the medical guidewire.

In another example, the number of the plurality of constituent wires of the braided wire can vary along the length of the medical guidewire.

In another example, the coiled wire first tubular member or at least one of the constituent wires of the braided wire second tubular member can include radiopaque material. The radiopaque material can include a tungsten wire.

In another example, a medical guidewire can further include a polymer layer applied to the braided wire second tubular member.

In another example, the medical guidewire can further include a hydrophilic coating on the braided wire second tubular member.

In another example, the coiled wire first tubular member can include stainless steel.

In another example, the coiled wire first tubular member can include a nickel-titanium alloy.

In another example, the medical guidewire can further include a laser cut metallic tube extending along at least a portion of the length of the medical guidewire, wherein at least a portion of the coiled wire first tubular member and at least a portion of the braided wire second tubular member are disposed within the laser cut metallic tube.

In some aspects, the techniques described herein relate to a medical guidewire that includes: a coiled wire first tubular member having a plurality of coils and openings between adjacent coils of the plurality of coils and extending along a length of the medical guidewire; and an inflatable balloon that is configured for radial expansion in a direction transverse to an axis along the length of the medical guidewire at which the inflatable balloon is located

Implementations can include one or more of the following features, alone, or in any combination with each other.

For example, the medical guidewire can further include: a braided wire second tubular member including a plurality of constituent wires braided together and openings between the constituent wires that are braided together, the braided wire second tubular member extending along the length of the medical guidewire.

In another example, the medical guidewire can further include a side-emitting optical fiber extending along the length of the medical guidewire.

In another example, the medical guidewire can further include holes configured for providing fluid pressure to expand the inflatable balloon in the direction transverse to the axis along the length of the medical guidewire at which the inflatable balloon is located.

### BRIEF DESCRIPTION OF THE DRAWINGS

FIG. 1 is a schematic diagram of a medical device that may be used in surgical procedures.

FIGS. 2A and 2B are schematic cross-sectional views of an example guidewire that can be used to navigate through a pathway in a patient's body to a site for a medical procedure.

FIG. 3 is a schematic diagram of an example guidewire that includes a coiled metallic wire, a metallic braid, and a side-emitting optical fiber.

FIGS. 4A and 4B are perspective views of stiffener tubes that can be used to stiffen a guidewire.

FIG. 5 is a schematic diagram of an example side view of a guidewire that includes a coiled metallic wire, a metallic braid surrounding the coiled metallic wire, a laser cut metal tube surrounding the metallic braid, and a side-emitting optical fiber disposed within the tubular structure of the guidewire.

FIGS. 6A and 6B are schematic diagrams of a side view of a guidewire that includes a portion that is configured to expand radially under the control of an operator or physician.

FIG. 7 is a schematic diagram of a system with which a pressure of a balloon on the guidewire can be controlled.

### DETAILED DESCRIPTION

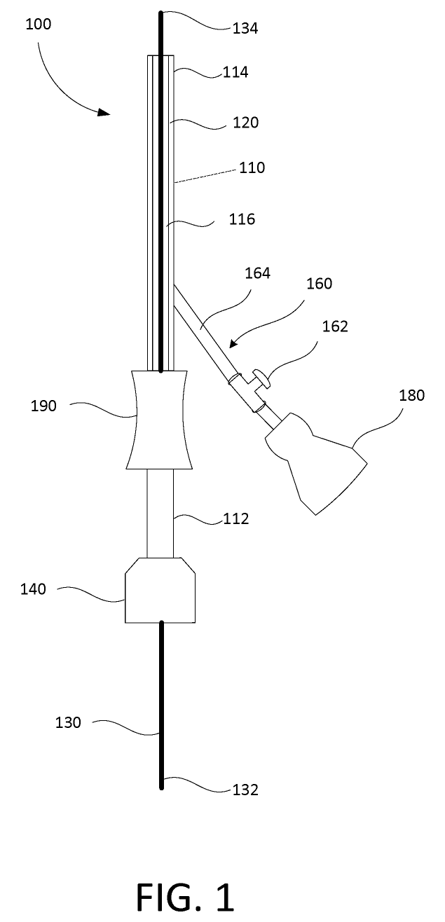

FIG. 1 is a schematic diagram of a medical device 100 that may be used in surgical procedures. For example, in some implementations, a portion of the medical device 100 may be placed within the body of a patient to create an access, or port, for further medical procedures. More specifically, in some implementations, a portion of the medical device 100 may be located within the body of the patient such that a distal end portion of the medical device 100 is disposed within the body of the patient, and a proximal end portion of the medical device 100 may be located outside the body of the patient. In some implementations, an additional or secondary medical device or a medical instrument may be inserted into the body of the patient via the medical device 100 to perform an additional medical procedure.

In the illustrated implementation, the medical device 100 can include a guidewire 130 and an elongated member 110. The guidewire 130 (e.g., a medical guidewire) is a flexible wire structure that is used to navigate and access the inside of the patient’s body during a medical procedure. The elongated member 110 may have a tubular structure, so that the elongated member can slide over the guidewire 130 into a position that has been accessed by the guidewire. In this manner, the relatively flexible guidewire 130 can be used to navigate through tortuous pathways (e.g., arteries, veins, ducts, tracts, and the like) in a patient’s body to reach a site at which a medical treatment or procedure is to be performed, and then the elongated member 110 can be slid over the guidewire to follow the pathway previously navigated by the guidewire 130 to reach the site.

The elongated member 110 includes a first or proximal end portion 112 and a second or distal end portion 114, and the guidewire includes a first or proximal end portion 132 and a second or distal end portion 134. The elongated member 110 defines one or more lumens 116 that extend from the first or proximal end portion 112 to the second or distal end portion 114, and at least one of the lumens 116 can slide over the guidewire 130. The elongated member 110 includes a sidewall 120 that has an inner surface and an outer surface, and the inner surface can define a lumen 116. In some implementations, the elongated member 110 is linear or substantially straight or linear. In other implementations, the elongated member 110 is curved or is flexible and may form a curve or have a curved portion. The elongated member 110 or the sidewall 120 may have a non-circular cross-sectional shape. For example, elongated member 110 or the sidewall 120 may have a round or circular cross-sectional shape. In yet other implementations, the elongated member 110 or the sidewall 120 has a different cross-sectional shape, such as square, rectangular, triangular, or any other shape.

The medical device 100 can include a side port 160, and the side port 160 can define a lumen 164. The side port 160 can be coupled to the elongated member 110 or the sidewall 120 such that the lumen 164 of the side port 160 is in fluid communication with the lumen 116 defined by the elongated member 110 or the sidewall 120. The side port 160 is configured to pass fluid into, and receive fluid from, the lumen 116 defined by the elongated member 110 or the sidewall 120. In some implementations, the side port 160 can include a valve 162 that is disposed within the lumen 164 defined by the side port 160. The valve 162 can be configured to help regulate or control the fluid flow through the lumen 164 defined by the side port 160.

The medical device 100 can include a collection member 180 that is coupled to the side port 160 and that is configured to collect material that is disposed within the fluid that flows through or within the lumen 164 of the side port 160. For example, the collection member 180 may be a filter that is configured to collect kidney stones or kidney stone fragments that pass from the body of the patient and through the lumen 164 of the side port 160.

In the illustrated implementation, the medical device 100 includes a handle member or handle portion 190. The handle member or handle portion 190 is coupled to the elongated member 110 or the sidewall 120. The handle member or handle portion 190 is configured to be grasped by a physician or other medical practitioner to place the medical device 100 within the body of a patient or during the use of the medical device 100.

In use, the distal end 134 of the guidewire 130 may be inserted into the body of a patient and steered through pathways within the body to a site at which a medical procedure is to occur. The elongated member 110 can follow the path of the guidewire to the site and can serve as a conduit to deliver one or more tools, fluids, medications, etc., to the site with which at to carry out the procedure. In some implementations, a portion of the medical device 100 may be inserted into the body of the patient such that the second or distal end portion 114 of the elongated member 110 is disposed within the body of the patient, and the first or proximal end portion 112 of the elongated member 110 is disposed outside of the body of the patient (or extends from the body of the patient). For example, in some implementations, the medical device 100 may be placed within the body of the patient such that the second or distal end portion 114 is disposed within a kidney or ureter or cystic duct of the patient. In other implementations, the elongated member 110 of medical device 100 can be placed within the body of the patient such that the second or distal end portion 114 is disposed at a different location within the body of the patient, for example, in the common bile duct, pancreatic duct, cystic duct, liver, gallbladder, or pancreas of the patient.

The medical device 100 can include an end cap, or a working channel cap, 140 located at the proximal end portion 112 of the elongated member 110. The working channel cap 140 can form a seal at the proximal end portion 112 of the elongated member 110 to maintain a fluid pressure within a lumen 116 of the elongated member 110. The working channel cap 140 can allow an additional or secondary medical device or medical instrument to be inserted into a lumen 116 of the elongated member 110 to deliver the additional or secondary medical device or medical instrument to the site at which the procedure is to be performed.

For example, in some implementations, an elongated member secondary medical device may be inserted into a lumen defined by the elongated member 110 such that the elongated member secondary medical device is disposed within the lumen. Accordingly, the elongated member secondary medical device may be used to perform additional medical procedures once such device is disposed within the body of the patient. In some implementations, the elongated member secondary medical device can be a scope. In other implementations, the elongated member secondary medical device can be another type of medical device.

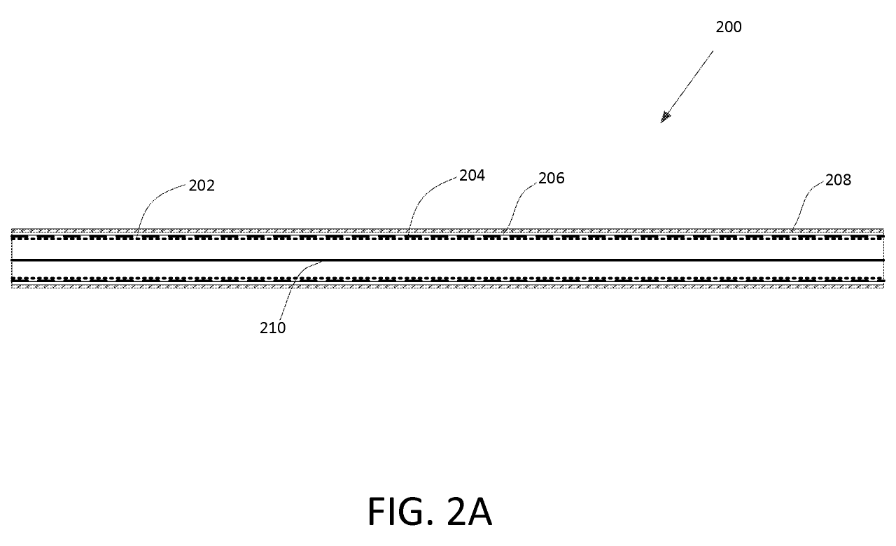

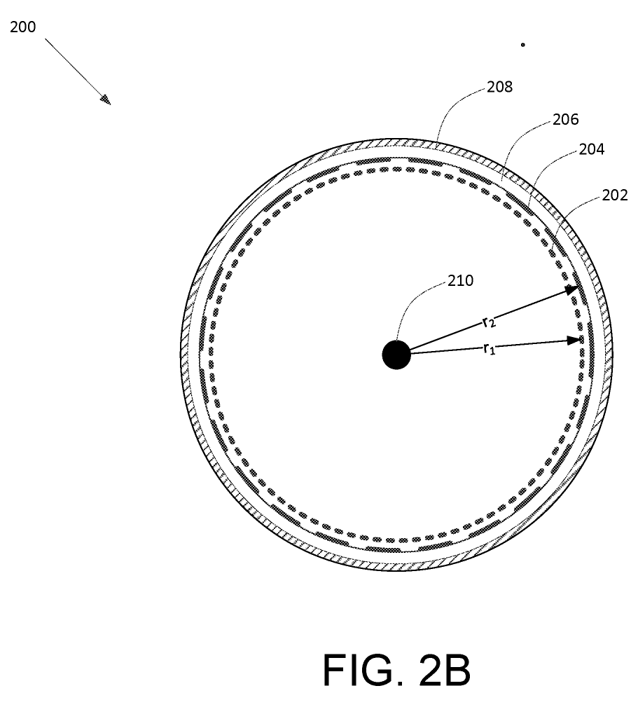

FIGS. 2A and 2B are schematic cross-sectional views of an example guidewire 200 that can be used to navigate through a pathway in a patient’s body to a site for a medical procedure. The cross-sectional view of FIG. 2A is along an axis of the elongated member. FIGS. 2B is a schematic cross-sectional view of the example guidewire 200 with the view being perpendicular to the axis of the elongated member. In some implementations, the guidewire 200 can include a coiled metallic wire 202. For example, the coiled metallic wire 202 can have a diameter and can be coiled into a generally tubular shape with a plurality of coils having a radius, r₁, extending along a length of the coiled metallic wire 202. In some implementations, the radius of the coiled metallic wire 202 can be greater than ten times the diameter of the wire that is coiled into the generally tubular shape of the coiled metallic wire 202. In some implementations, the diameter of the coiled metallic wire 202 can be less than 2 mm. The coiled metallic wire 202 can include, for example, stainless steel or a nickel-titanium alloy (e.g., Nitinol®). In some implementations, adjacent coils of the coiled metallic wire 202 may not touch, such that openings may exist between the adjacent coils.

In some implementations, the guidewire 200 can include a metallic braid 204. For example, the metallic braid 204 can include a plurality of constituent metallic wires that are braided together into a generally tubular shape having a radius r₂ extending along the length of the guidewire 200, where the radius of the metallic braid 204 is larger than the radius of the coiled metallic wire 202. In some implementations, the diameter of the metallic braid 204 can be less than 2 mm, less than 1.5 mm, or less than 1 mm. The metallic braid 204 can include, for example, stainless steel or a nickel-titanium alloy (e.g., Nitinol®). The metallic braid 204 can fit over the coiled metallic wire 202, and in some implementations, the coiled metallic wire 202 can be used as mandrel on which the metallic braid 204 is created with a braiding apparatus. In some implementations, the braided constituent wires of the metallic braid may be braided such that openings exist between the adjacent the constituent wires.

The combination of the coiled metallic wire 202 and the metallic braid 204 can define a generally tubular shape of the guidewire 200 with a sidewall defined by the coiled metallic wire 202 and the metallic braid 204. The diameters of the wires used in the coiled metallic wire 202 and in the metallic braid 204 can be selected, such that a diameter of the sidewall is less than 0.2 mm thick, less than 0.1 mm thick, or less than 0.5 mm thick.

In some implementations, the guidewire 200 can include an outer jacket 206 that includes a polymer material layer and that surrounds the coiled metallic wire 202 and the metallic braid 204. The outer jacket 206 has an outer sidewall that defines an outer diameter or maximum cross-section of the elongated member. The outer jacket 206 can include PTFE or can include a non-PFAS material. For example, the outer jacket 206 can include Polyphtalamide, Polyetheretherketone, Polyphenylensulfide, Polyamide, Polyolefin, Polyurethane, or other non-PFAS materials. The outer jacket 206 of the guidewire 200 can be extruded over the coiled metallic wire 202 and the metallic braid 204 in an extrusion process. In some example implementations, polymer material layer can be heated to a temperature above a glass temperature (T_{g}) of the polymer material, so that the polymer material layer can flow and be formed into the shape of the outer jacket 206. In some implementations, the guidewire 200 can include a hydrophilic coating 208 on the outside of the outer jacket 206 or directly the coiled metallic wire 202 and/or on the metallic braid 204. The outer jacket 206 and the hydrophilic coating 208 can be transparent to light.

In some implementations, the guidewire 200 can include a side-emitting optical fiber 210. The side-emitting optical fiber 210 can include a transparent material for guiding light along a length of the fiber. For example, the side-emitting optical fiber 210 can include a central core and a cladding surrounding the core, where the index of refraction of the cladding is less than the index of refraction of the core, such that light that traverses the core at angles below a threshold angle relative to a longitudinal axis of the fiber is totally internally reflected at a smooth interface between the core and the cladding. In some implementations, the interface between the core and the cladding can be created intentionally to include deviations from smoothness, such that a proportion of light travelling in the optical fiber is scattered in a radial direction perpendicular to the length of the optical fiber and out of the fiber in a radial direction. Because of the openings in the coiled metallic wire 202 and in the metallic braid 204 and because of the transparency of the outer jacket 206 and the hydrophilic coating 208, light emitted from the side-emitting optical fiber 210 in a radial direction perpendicular to the longitudinal axis of the fiber can exit the guidewire 200 to illuminate an environment around the guidewire.

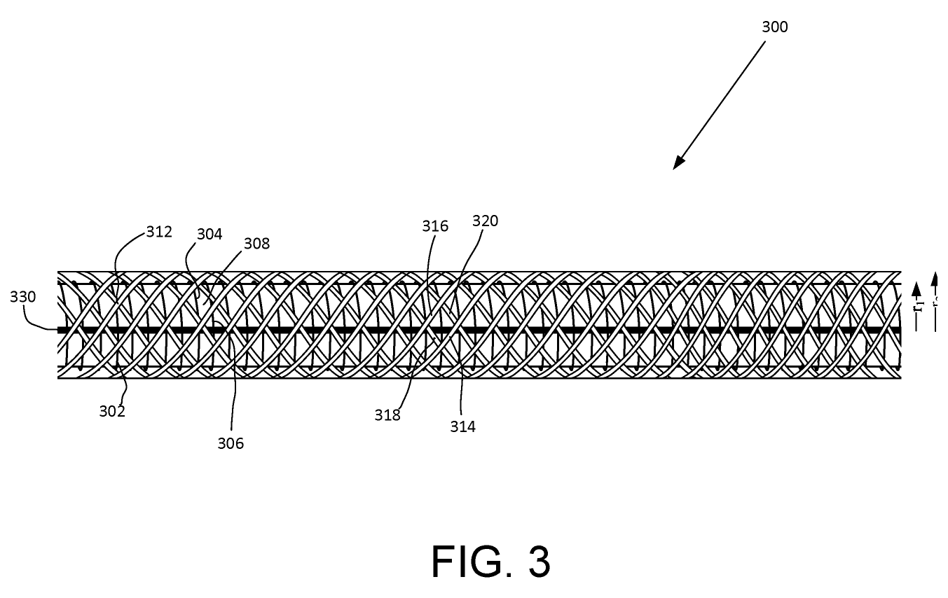

FIG. 3 is a schematic diagram of an example guidewire 300 that includes a coiled metallic wire 302, a metallic braid 312, and a side-emitting optical fiber 330. The coiled metallic wire 302 can include a metal, such as, for example, stainless steel, a nickel-titanium alloy (e.g., Nitinol®) and can have a diameter (e.g., less than 0.1 mm, less than 0.05 mm, or less 0.02 mm). The wire 302 can be coiled into a helix having a plurality of coils, with a radius, r₁, such that the plurality of coils define a generally tubular structure having a diameter that is greater than the diameter of the wire that is coiled. In some implementations, adjacent coils, for example, coils 304, 306 of the coiled metallic wire 302 may not touch, such that openings exist between the adjacent coils. For example, an opening 308 exists between adjacent coils 304 and 306. When a side-emitting optical fiber is located within the helix of the coiled metallic wire 302, and light is emitted radially from the optical fiber, the light can pass through the openings to illuminate an area around the coiled metallic wire 302. The distance between the centers of adjacent coils can be referred to as the “pitch” of the coil and can be used to parameterize the coil.

The stiffness of the coiled metallic wire 302 can be affected by a number of parameters, including the type of metal used in the wire 302, the diameter of the coils, the diameter of the coil wire, and the pitch of the coiled metallic wire 302. For example, the coiled metallic wire 302 can be stiffer when a thicker wire is used to form the coils than when a thinner wire is used to form the coils; the coiled metallic wire 302 can be stiffer when a stainless steel material is used for the wire than when a Nitinol® material is used for the wire; the stiffness of the coiled metallic wire 302 can increase with increasing diameter of the coils; and the stiffness of the coiled metallic wire usually can increase as the pitch of the coils increases.

In some implementations, the value(s) of one or more parameter affecting the stiffness of the coiled metallic wire 302 can vary along a length of the guidewire 300, so that the guidewire can have a stiffness that varies along its length. For example, the pitch of the coils of the coiled metallic wire 302 can vary along the length of the guidewire to vary a stiffness of the guidewire 300. In some implementations, the pitch of the coils can be lower near a distal end of the guidewire 300 than it is at a proximal end of the guidewire, so that the distal end is relatively flexible and able to navigate tortuous pathways, while the proximal end has greater stiffness and pushability to allow a physician to push the guidewire through the patient’s body. In some implementations, the coiled metallic wire 302 can have a first, relatively low, pitch near the distal end of the guidewire 300 and a second, relatively high pitch in other parts of the guidewire. In some implementations, the coiled metallic wire 302 can have a pitch that decreases monotonically in a region near the distal end of the guidewire 300.

In some implementations, the thickness (e.g., diameter) of the wire that is coiled into the coiled metallic wire 302 can vary along the length of the guidewire 300 to vary a stiffness of the guidewire 300. In some implementations, the thickness of the wire can be lower near a distal end of the guidewire 300 than it is at a proximal end of the guidewire, so that the distal end is relatively flexible and able to navigate tortuous pathways, while the proximal end has greater stiffness and pushability to allow a physician to push the guidewire through the patient’s body. In some implementations, the wire of the coiled metallic wire 302 can have a first, relatively low, thickness near the distal end of the guidewire 300 and a second, relatively high, thickness in other parts of the guidewire. In some implementations, the coiled metallic wire 302 can have a thickness that decreases monotonically in a region near the distal end of the guidewire 300. The thickness of the wire can be varied by a variety of methods, including chemical etching of the wire of the guidewire 300. For example, the wire of the coiled metallic wire 302 and/or the metallic braid 312 can be chemically etched by hydrochloric acid, nitric acid, sulfuric acid, etc. to remove metal from the wire to reduce thickness of the wire. The thickness of the wire can also vary along the length by sequentially attaching coils of different coiled metallic wire 302 diameters. In some implementations, the wire of the coil metallic wire 302 can have a first, relatively low, thickness near the distal end for the guidewire 300 and a second, relatively high, thickness in other parts of the guidewire.

The metallic braid 312 can include a plurality of constituent metal wires that are woven together to form a braided metal tubular structure, and the constituent wires metal, such as, for example, stainless steel, a nickel-titanium alloy (e.g., Nitinol®). The constituent wires can be braided into a tubular shape having a plurality of coils, with a radius, r₂. In some implementations, adjacent parallel constituent wires for example, wires 314, 316 and wires 318, 320 may be spread apart, such that openings may exist between the constituent wires of the metallic braid 312. For example, an opening exists between constituent wires 314, 316, 318, 320. When the side-emitting optical fiber 330 is located within the metallic braid 312, and light is emitted radially from the optical fiber, the light can pass through the openings to illuminate an area around the metallic braid 312.

The distance along a length of the metallic braid 312 between the centers of crossings of constituent wires can be referred to as the “pick” of the braid and can be used to parameterize the braid. For example, the distance between the center of the crossing of wires 314and 316 and the center of the crossing of wires 318 and 320 along the length of the braid can define the pick of the braid. The density of picks in the metallic braid 312 (e.g., the number of picks per inch (PPI) along the length of the braid) can be used to characterize the stiffness of the braid.

Thus, the stiffness of the metallic braid 312 can be affected by a number of parameters, including the type of metal used in the constituent wires, the diameter of the braid, the diameter of the wires, the number of constituent wires in the braid, and the PPI of the braid. For example, the metallic braid 312 can be stiffer when thicker constituent wires are used to form the metallic braid than when a thinner wire is used to form the braid; the metallic braid 312 can be stiffer when a stainless steel material is used for the constituent wires than when Nitinol® material is used for the wires; the stiffness of the metallic braid 312 can increase with increasing diameter of the coils; the stiffness of the metallic braid 312 can increase with an increasing number of constituent wires in the braid; and the stiffness of the metallic braid 312 can increase as the PPI of the braid decreases.

In some implementations, the value(s) of one or more parameter affecting the stiffness of the metallic braid 312 can vary along a length of the guidewire 300, so that the guidewire can have a stiffness that varies along its length. For example, the PPI of the metallic braid 312 can vary along the length of the guidewire to vary a stiffness of the guidewire 300. In some implementations, the PPI of the metallic braid 312 can be higher near a distal end of the guidewire 300 than it is at a proximal end of the guidewire, so that the distal end is relatively flexible and able to navigate tortuous pathways, while the proximal end has greater stiffness and pushability to allow a physician to push the guidewire through the patient’s body. In some implementations, the metallic braid 312 can have a first, relatively high, PPI near the distal end of the guidewire 300 and a second, relatively low, pitch in other parts of the guidewire. In some implementations, the metallic braid 312 can have a PPI that increases monotonically in a region near the distal end of the guidewire 300.

In another example, the number of constituent wires in the metallic braid 312 of the guidewire 300 can vary along the length of the guidewire. For example, the metallic braid 312 can be constructed to include a higher number of constituent wires (e.g., 32 wires) at the proximal end and along most of the length of the guidewire 300 and a lower number of constituent wires (e.g., 16 wires) at the distal end, so that the metallic braid has a higher flexibility at its distal end than in the rest of the braid of the guidewire.

In some implementations, the thickness (e.g., diameter) of the constituent wires of the metallic braid 312 can vary along the length of the guidewire 300 to vary a stiffness of the guidewire 300. In some implementations, the thickness of the constituent wires can be lower near a distal end of the guidewire 300 than at a proximal end of the guidewire, so that the distal end is relatively flexible and able to navigate tortuous pathways, while the proximal end has greater stiffness and pushability to allow a physician to push the guidewire through the patient’s body. In some implementations, the constituent wires of the metallic braid 312 can have a first, relatively low, thickness near the distal end of the guidewire 300 and a second, relatively high, thickness in other parts of the guidewire. In some implementations, the wires of the metallic braid 312 can have a thickness that decreases monotonically in a region near the distal end of the guidewire 300. The thickness of the wires can be varied by a variety of methods, including chemical etching of the wires of the guidewire 300, for example with hydrochloric acid, nitric acid, sulfuric acid, etc. to remove metal from the wires to reduce thickness of the wires.

In some implementations, one or more constituent wires of the metallic braid 312 can include a radiopaque material (e.g., tungsten) to enhance the visibility of the metallic braid in an X-ray image. Thus, when the guidewire 300 is used during a fluoroscopy procedure, a physician can monitor the position of the guidewire in the patient’s body as the guidewire is advanced through a pathway in the patient’s body.

In some implementations, one or more constituent wires of the metallic braid 312 can include a side-emitting optical fiber, so that light emitted from the side-emitting optical fiber is emitted from the outside of the metallic braid 312, rather than from with the lumen defined by the metallic braid. The side-emitting optical fiber can be woven into the metallic braid during the braiding process used to create the metallic braid.

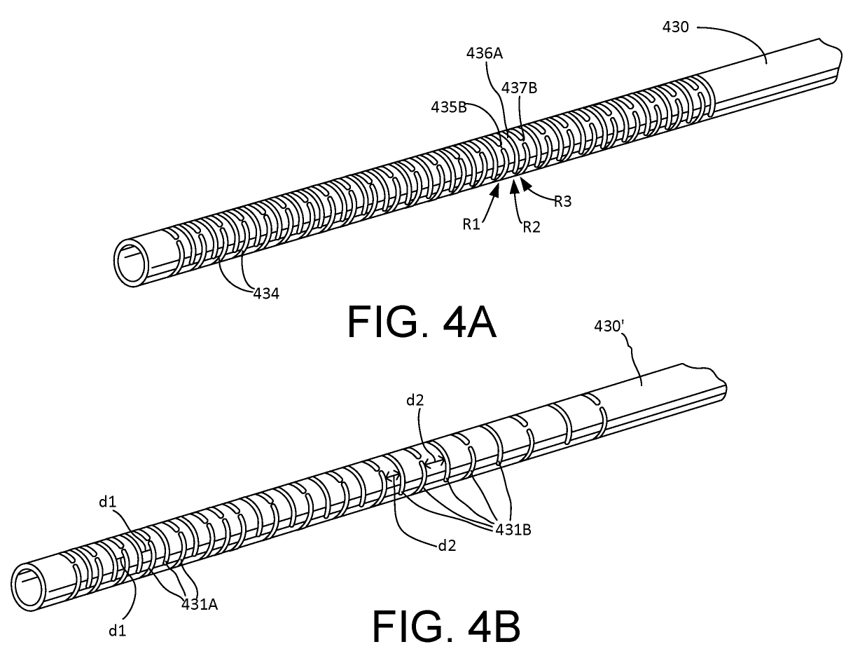

In some implementations, a portion of a guidewire that includes a coiled metallic wire and a metallic braid can additionally include a stiffener tube arranged to provide additional stiffness to the guidewire. FIGS. 4A and 4B are perspective views of stiffener tubes that can be used to stiffen a guidewire.

The stiffener tube 430 in the embodiment illustrated in FIG. 4A is a metal tube that has a plurality of slots 434 formed therein (preferably laser cut into the tube) to increase the flexibility of the tube. Each slot extends around a portion of the circumference of the tube 430 for less than 360 degrees (e.g., less than 180 degrees). Additionally, the slots can be staggered such that a solid portion of the tube between the space between slots in one row is adjacent a slotted portion of another row. Three rows of slots are numbered in FIG. 4A to illustrate how a slot portion 436A of row R2 is adjacent to a gap 435B (solid tube portion) between slot portions of row R1 and is to adjacent a gap 437B (solid tube portion) between slot portions of row R3. In some implementations, the amount of circumferential solid material of the metal tube between the slot ends can be varied to create a change in the stiffness of the tube. In some implementations, the slots can be arranged in a helical pattern on the tube, with the helical pattern having a clocking angle so that the spine appears to form a spiral. The spiral or helical pattern can be useful in tracking the location of the tube as it is inserted into the patient’s body.

As shown, the axial spacing between the slots in FIG. 4A is substantially equal. However, it is also contemplated that the spacing between the slots can be varied at various portions along the tube to provide areas of different flexibility. For example, in the embodiment of FIG. 4B, the slots of tube 430′ vary such that slots 431A at the distal portion of the tube 430′ are closer together (have a shorter distance between them, d1) than the slots 431B of a more proximal portion which have a greater distance between them, d2, to provide more flexibility at the distal end. Various slot spacings are contemplated. For example, the slots can be varied such that they become progressively further apart in a proximal direction or discrete regions of the tube can have slots of substantially equal spacing, but different than other regions of the tube.

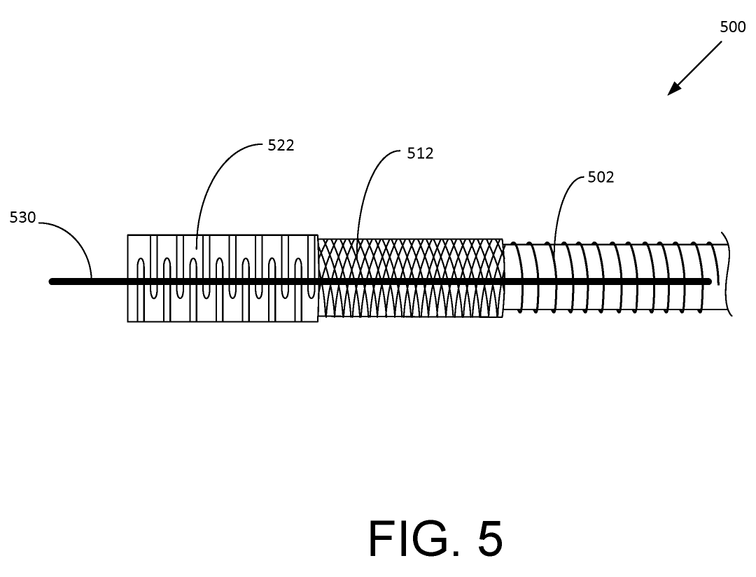

FIG. 5 is a schematic diagram of an example side view of a guidewire 500 that includes a coiled metallic wire 502, a metallic braid 512 surrounding the coiled metallic wire, a laser cut metal tube 522 surrounding the metallic braid 512, and a side-emitting optical fiber 530 disposed within the tubular structure of the guidewire (i.e., within a lumen formed by the coiled metallic wire 502). Each of the coiled metallic wire 502, the metallic braid 512, and the laser cut metal tube 522 include openings through which light from the side-emitting optical fiber 530 can pass, so that light emitted in the radial direction from the side-emitting optical fiber can illuminate an environment in a radial direction around the guidewire 500 (i.e., perpendicular to a longitudinal axis of the guidewire). For clarity, portions of the coiled metallic wire 502, the metallic braid 512, and the laser cut metal tube 522 are shown without showing the structures contained within them, except for the side-emitting optical fiber 530. The coiled metallic wire 502, the metallic braid 512, and the laser cut tube 522 need not be arranged in the radial order shown in FIG. 5, and in some implementations, the coiled metallic wire 502, the metallic braid 512, and the laser cut tube 522 can be arranged in a different radial order, for example, with the metallic braid 512 being surrounded by the coiled metallic wire 502, or with the laser cut tube 522 being surrounded by the metallic braid 512.

Guidewires, as described herein, can be used to cannulate a passageway in a patient’s body, where cannulation is the passage of a guidewire, catheter, or other device through a passageway of the body. For example, a guidewire can be passed through a duodenoscope and into the bile duct, cystic duct, or pancreatic duct during an initial step of an endoscopic retrograde cholangiography and pancreatography (ERCP) procedure, and then additional catheters can be passed over the guidewire to perform other steps of the ERCP procedure. For example, a spy scope can be passed over the guidewire after the guidewire has cannulated the passageway in the body. However, in some cases, the relative size of the spy scope or other device compared to the size of the guidewire and/or the tortuous anatomy of the ducts may make it difficult for the additional catheters (e.g., the spy scope) to cannulate the passageway even after the guidewire has cannulated the passageway.

Therefore, the guidewire can include a portion that can be configured for radial expansion (i.e., in a direction transverse to an axis along a length of the guidewire) to apply pressure to the tissue of the passageway surrounding the guidewire to expand the cross-sectional area (e.g., diameter) of the passageway to allow an additional catheter to more easily pass through the expanded passageway. Because the tissue may retain its shape for a period of time (e.g., tens of seconds) after the pressure has been removed, the portion of the guidewire can exert radial pressure on the tissue to expand the tissue and then cease exerting radial pressure to allow the other catheter to pass through the opening that has been expanded by the portion of the guidewire.

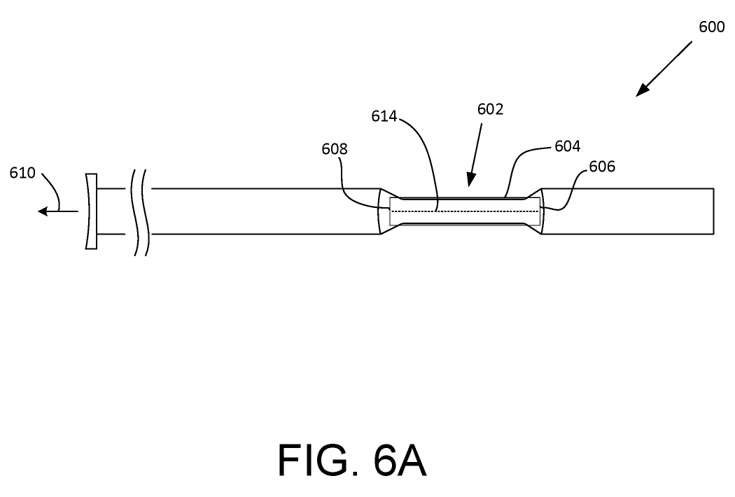

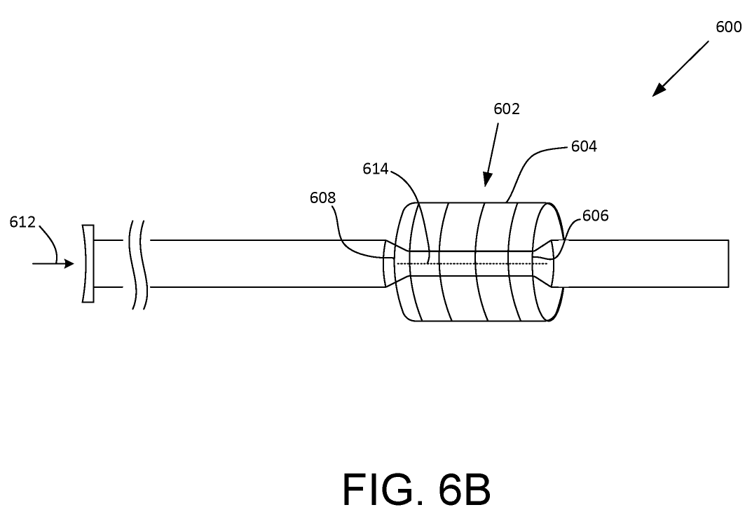

FIGS. 6A and 6B are schematic diagrams of a side view of a guidewire 600 that includes a portion 602 that is configured to expand radially under the control of an operator or physician. FIG. 6A shows the portion 602 in its unexpanded shape, and FIG. 6B shows the portion in its expanded shape.

In some implementations, the portion 602 can include an expandable balloon 604 that can be inflated by the application of fluid (e.g., air or liquid) pressure to the balloon. For example, in some implementations, the ballon can include a cylinder of elastic material that can be sealed at a first end 606 and at a second end 608 against a tubular portion of the guidewire (e.g., an airtight polymer jacket of the guidewire). Fluid pressure to inflate, and to deflate, the balloon 604 can be introduced through small holes or pores 614 in a sidewall of the guidewire (e.g., in a sidewall of the polymer jacket of the guidewire). The fluid pressure can be controlled by a physical or operator from outside the body of the patient to increase a fluid pressure in the balloon 604, as shown by the arrow 612 in FIG. 6B or to decrease an fluid pressure in the balloon 604, as shown by the arrow 610 in FIG. 6A. In some implementations, the wall thickness of the polymer jacket can have sufficient thickness and sufficient strength to withstand the fluid pressure that is used to inflate the balloon 604. In some implementations, in which the guidewire includes a coiled metallic wire and/or a metallic braid, the coiled metallic wire and/or the metallic braid can be embedded in the polymer jacket, at least at the portion 602 that includes the expandable jacket and in the proximal portion upstream of the portion 602.

In some implementations, the guidewire can be tapered at the portion 602 of the guidewire, such that the portion 602 of the guidewire 600 can have a diameter that is smaller than other portions the guidewire, particularly other portions of the guidewire that are located within about 3 cm of the portion 602 along the length of the guidewire. In this manner, any increase in friction due to the presence of the balloon 604 at the portion may be mitigated by the decreased diameter of the portion compared to neighboring portions.

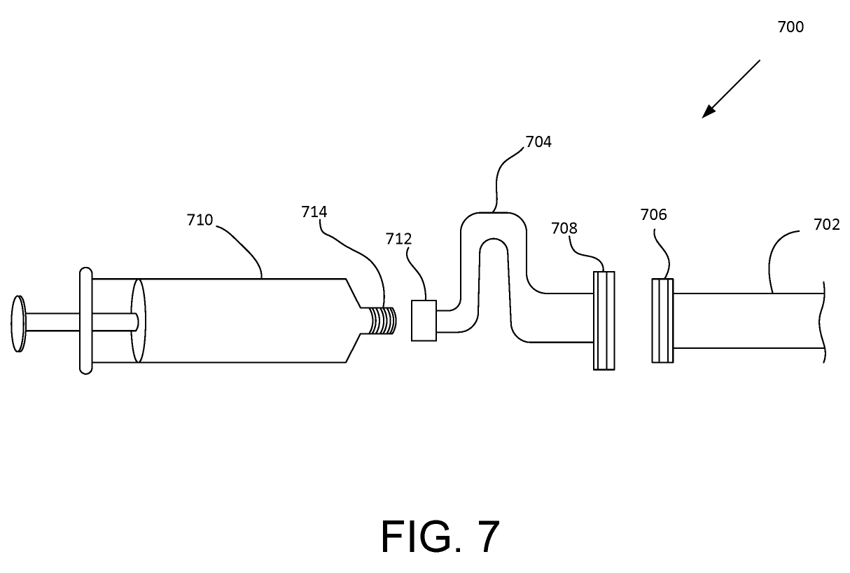

FIG. 7 is a schematic diagram of a system 700 with which the pressure of the balloon on the guidewire can be controlled. The guidewire 702 can be coupled, outside the patient’s body, to a flexible hose or pipe 704 by a first coupling member 706 on the guidewire and a second coupling member 708 on the flexible hose or pipe 704. The flexible hose or pipe 704 can be coupled to a pump 710 by a third coupling member 712 on the flexible hose or pipe 704 and a fourth coupling member 714 on the pump 710. In some implementations, the pump 710 can include a syringe that pumps fluid (e.g., air or liquid) to, and sucks fluid from, the balloon that is attached to the guidewire.

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some implementations, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations.

What is claimed is:

1. A medical guidewire comprising:
a coiled wire first tubular member having a plurality of coils and openings between adjacent coils of the plurality of coils and extending along a length of the medical guidewire;
a braided wire second tubular member including a plurality of constituent wires braided together and openings between the constituent wires that are braided together, the braided wire second tubular member extending along the length of the medical guidewire; and
a side-emitting optical fiber extending along the length of the medical guidewire.

2. The medical guidewire of claim 1, wherein the coiled wire first tubular member is disposed within the braided wire second tubular member.

3. The medical guidewire of claim 1, wherein the side-emitting optical fiber is disposed within the coiled wire first tubular member.

4. The medical guidewire of claim 1, wherein the constituent wires of the braided wire second tubular member include the side-emitting optical fiber.

5. The medical guidewire of claim 1, wherein the side-emitting optical fiber includes a core and a cladding surrounding the core, wherein an interface between the core and the cladding includes deviations from smoothness that are configured to scatter light out of the optical fiber in a radial direction transverse to a longitudinal axis of the side-emitting optical fiber.

6. The medical guidewire of claim 1, wherein the coiled wire first tubular member has a pitch that varies along the length of the medical guidewire.

7. The medical guidewire of claim 1, wherein the coiled wire first tubular member has a thickness that varies along the length of the medical guidewire.

8. The medical guidewire of claim 1, wherein the braided wire second tubular member has a picks per inch (PPI) that varies along the length of the medical guidewire.

9. The medical guidewire of claim 1, wherein the number of the plurality of constituent wires of the braided wire can vary along the length of the medical guidewire.

10. The medical guidewire of claim 1, wherein the coiled wire first tubular member or at least one of the constituent wires of the braided wire second tubular member includes radiopaque material.

11. The medical guidewire of claim 10, wherein the radiopaque material includes a tungsten wire.

12. The medical guidewire of claim 1, further comprising a polymer layer applied to the braided wire second tubular member.

13. The medical guidewire of claim 12, further comprising a hydrophilic coating on polymer layer applied to the braided wire second tubular member.

14. The medical guidewire of claim 1, wherein the coiled wire first tubular member includes stainless steel.

15. The medical guidewire of claim 1, wherein the coiled wire first tubular member includes a nickel-titanium alloy.

16. The medical guidewire of claim 1, further comprising a laser cut metallic tube extending along at least a portion of the length of the medical guidewire, wherein at least a portion of the coiled wire first tubular member and at least a portion of the braided wire second tubular member are disposed within the laser cut metallic tube.

17. A medical guidewire comprising:
a coiled wire first tubular member having a plurality of coils and openings between adjacent coils of the plurality of coils and extending along a length of the medical guidewire; and
an inflatable balloon that is configured for radial expansion in a direction transverse to an axis along the length of the medical guidewire at which the inflatable balloon is located.

18. The medical guidewire of claim 17, further comprising:
a braided wire second tubular member including a plurality of constituent wires braided together and openings between the constituent wires that are braided together, the braided wire second tubular member extending along the length of the medical guidewire.

19. The medical guidewire of claim 17, further comprising:
a side-emitting optical fiber extending along the length of the medical guidewire.

20. The medical guidewire of claim 17, further comprising: holes configured for providing fluid pressure to expand the inflatable balloon in the direction transverse to the axis along the length of the medical guidewire at which the inflatable balloon is located.
